(19) United States
(12) Patent Application Publication
(10) Pub. No.: US 2004/0028687 A1
(43) Pub. Date: Feb. 12, 2004

(54) Methods and compositions for the targeted delivery of therapeutic substances to specific cells and tissues

(76) Inventor: Ernst Rudolf Waelti, Muenchenbuchsee (CH)

Correspondence Address: PERKINS COIE LLP, POST OFFICE BOX 1208, SEATTLE, WA, 98111-1208, US

(21) Appl. No.: 10/346,044

(22) Filed: Jan. 15, 2003

Related U.S. Application Data
(60) Provisional application No. 60/349,609, filed on Jan. 15, 2002.

(51) Int. Cl. A61K 39/395; C07K 16/46

(52) U.S. Cl. 424/178.1; 530/391.1

(57) ABSTRACT

Provided are methods and compositions for the targeted delivery of therapeutic substances to specific cells and tissues. The methods and compositions of the present invention can be adapted for a wide variety of therapeutic applications that benefit from cell or tissue-specific delivery of drugs, thereby increasing the therapeutic index of drugs that otherwise produce systemic toxicity.

### STATEMENT OF PRIORITY

This application claims priority to U.S. Provisional Patent Application Serial No. 60/349,609 filed on Jan. 15, 2002.

### FIELD OF THE INVENTION

The present invention relates to the fields of biochemistry, molecular biology, and immunology. Specifically, the invention relates to highly targeted synthetic membrane vesicles for the delivery of therapeutic substances to selected cells and tissues, as well as their production.

### BACKGROUND OF THE INVENTION

Various publications or patents are referred to in parentheses throughout this application to describe the state of the art to which the invention pertains. Each of these publications or patents is incorporated by reference herein.

One of the basic goals of chemical and gene therapy is to deliver a therapeutic substance efficiently and specifically to a site of disease. Some substances may be delivered in free form whereas others require a carrier in order to reach and enter their final destination because of rapid clearance from the area of introduction or the circulation, obstruction by biological barriers which they cannot penetrate, or because they either produce systemic toxicity or are highly immunogenic. Since their discovery almost 40 years ago (Bangkam et al., J. Mol. Biol. 13, 238-252, 1965), liposomes have been widely used as carriers for drug and gene delivery. These closed spherical vesicles allow the insertion of lipophilic materials in the phospholipid bilayer and the encapsulation of hydrophilic compounds in the aqueous compartment. Because of their biodegradability and low toxicity, liposomes can be safely administered without serious side effects. Liposomes can alter the biodistribution of entrapped substances and protect the enclosed materials from inactivation by host defense mechanisms. To be effective as carriers, liposomes must be able to efficiently combine stability in the circulation with the ability to make the therapeutic compound bioavailable at the target site. While advances in the targeting and steric stabilization have led to improved tissue-selectivity and prolonged circulation time of liposomes, respectively, their tendency to remain in the extracellular environment represents a major drawback in therapeutic delivery (Yuan et al. Cancer Res. 54: 3352-3356, 1994). Because of the absence of general fusion peptides in liposome membranes, cellular uptake of unmodified liposomes, as well as that of antibody-targeted immunoliposomes, is dependent on the surface density of liposome-conjugated antibody and the target cell rate of receptor internalization (Kirpotin et al., Biochemistry; 36: 66-75, 1997).

Virosomes are modified liposomes that contain reconstituted fusion-active viral envelope proteins anchored in the phospholipid bilayer (Gluck et al., U.S. Pat. No. 6,040,167). The presence of the viral envelope proteins such as hemagglutinin (HA) allows the virosome envelope to attach to cell surface receptors and to enter the cell by receptor-mediated endocytosis. Like liposomes, virosomes can be used to deliver therapeutic substances to a wide variety of cells and tissues, but unlike liposomes, virosomes offer the advantage of efficient entry into the cells followed by the intracellular release of the virosomal contents triggered by the viral fusion protein. Moreover, due the incorporation of active viral fusion proteins into their membranes, virosomes release their contents into the cytoplasm immediately after being taken up by the cell, thereby preventing the degradation of the therapeutic substance in the acidic environment of the endosome (U.S. Pat. No. 6,040,167).

As with liposomes, targeting of virosomes to particular tissues can be achieved by conjugating cell-specific antibodies or ligands for cell surface receptors to the virosomal membrane. However, the construction of such targeted virosomes by previous methods presents technical difficulties and produces variable and often undesirable results, such as the precipitation of vesicles during the conjugation step which renders them useless for delivery applications. Additionally, even when the conjugation of a targeting ligand or antibody fragment to the virosomal membrane is accomplished, the binding site of the targeting moiety is often unavailable for binding to its cellular target, possibly due to steric hindrance by the hemagglutinin (HA) fusion proteins present in the virosomal membranes, or due to lack of control over the positioning of the targeting protein during chemical crosslinking of the targeting moiety to the virosomal membrane.

Thus, functional ligand- or antibody-conjugated virosomes with fully available targeting binding sites for efficient binding to specific target cells and tissues, as well as reliable methods of producing them would be a significant improvement in the targeted delivery of therapeutics.

### BRIEF DESCRIPTION OF THE DRAWINGS

FIG. 1 Shows that the virosomes are capable of binding to the tumor cell membranes and that Fab′-conjugated virosomes show increased binding to antigen expressing cells through specific binding via their surface anti-rNeu. Fab′ fragments.

Histograms represent rNeu⁺ and rNeu⁻ tumor cell lines stained with the specific rNeu-Ab followed by FITC-conjugated goat-anti mouse IgG (rNeu) or with an isotype matched primary Ab followed by FITC-conjugated secondary IgG (neg) (A+C). For virosome-binding assays, cells were incubated with either FITC-labeled virosomes conjugated with Fab′-fragments of anti-rNeu mAb (Fab′-Vir) or with unconjugated FITC-labeled virosomes (Vir) (B+D). Results are expressed as mean fluorescence intensity.

FIG. 2: Analysis of internalization in confocal laser scanning microscopy (CLSM). rNeu⁺ and rNeu⁻ tumor cells were stained with FITC-labeled anti-rNeu mAb (A+C) or FITC labeled Fab′-Vir (B+D, green) and rhodamine-labeled F-actin (red). 3D reconstruction: xy-projection (A′,B′,C′,D′), xz-projections (A″,B″,C″,D″). Arrows mark the position of projections.

FIG. 3: Antiproliferative activity of Fab′-Vir on rNeu⁺/rNeu³¹ tumor cells in vitro. rNeu⁺ (closed symbols) and rNeu⁻ (open symbols) tumor cells were cultured in monolayers for 48 h with Vir, Fab′-Vir and anti-rNeu mAb (7.16.4) at doses indicated. The proliferation of the cells after culture with the different compounds was measured by a colorimetric cytotoxicity assay (XTT). The results shown (with mean and SEM) represent 4 independent experiments. MAb clone 7.16.4 (□), Fab′-Vir (◯) and Vir (∇).

FIG. 4: Effect of virosome treatment on established rNeu+and rNeu⁻ tumors. 2×10⁶ rNeu⁺ tumor cells (A-C) were injected s.c. and treatment was started when tumor size had reached 5 mm in diameter. Therapeutic i.v. injections were performed every 3-4 days and tumor size was assessed twice a week by measuring length and width of each tumor with vernier calipers. Tumor volume was calculated using the formula π/6×largest diameter x (smallest diameter)² (A) Treatment with free Doxo and Doxo-Vir compared to control groups; (B) Doxo-Vir and Fab′-Doxo-Vir compared to control groups; (C) Fab′-Vir and Fab′-Doxo-Vir compared to control groups. These results show the mean and SEM. Control groups (◯), free Doxo (▾), Doxo-Vir (♦), Fab′-Vir (●), Fab′-Doxo-Vir (▪). (D) The treatment with injections of Fab′-Doxo-Vir, Doxo-Vir and free Doxo (all 150 μg/ml doxorubicin) was evaluated in mice with established rNeu⁻ tumors. Tumor sizes are shown at day 8 and 21 after tumor inoculation. Control groups (open column), Doxo-Vir (hatched column), Fab′-Vir (diagonal column), Fab′-Doxo-Vir (close column). These are results of 4-6 experiments each including 3-5 mice per group.

FIG. 5: Histological analysis of rNeu⁺ tumor injection site. Paraffin sections from tumor injection site at day 5 after injection of 2×10 rNeu⁺ tumor cell line (NF9006) into A) control animals, B) mice treated with Doxo-Vir, C) mice treated with Fab′-Vir and D) mice treated with Fab′-Doxo-Vir. Treatment was performed every 2^{nd} days.

FIG. 6: Effect of virosome treatment on recently implanted tumors. 2×10⁵ rNeu⁺ tumor cells were injected s.c. and treatment was started 3-5 days later. I.v. injections of the different virosome formulations such as Doxo-Vir, Fab′-Doxo-Vir, Fab′-Vir and free Doxo (all at a concentration of 150 μg/ml doxorubicin) were performed at the indicated time points (arrows) for a period of three weeks. Tumor size was assessed twice a week during therapy and weekly in the follow-up period. Tumor formation (defined as volume >90 mm³) in the different groups and the follow-up of >12 weeks is shown. Statistical analysis using Mann-Whitney rank test was performed. Control groups (◯), free Doxo (▾), Doxo-Vir (♦), Fab′-Vir (●), Fab′-Doxo-Vir (▪)

FIG. 7 shows the superior binding of conjugated virosomes to targeted cells as compared to liposomes.

FIG. 8 shows the quantification of Fab′ fragments in virosomes.

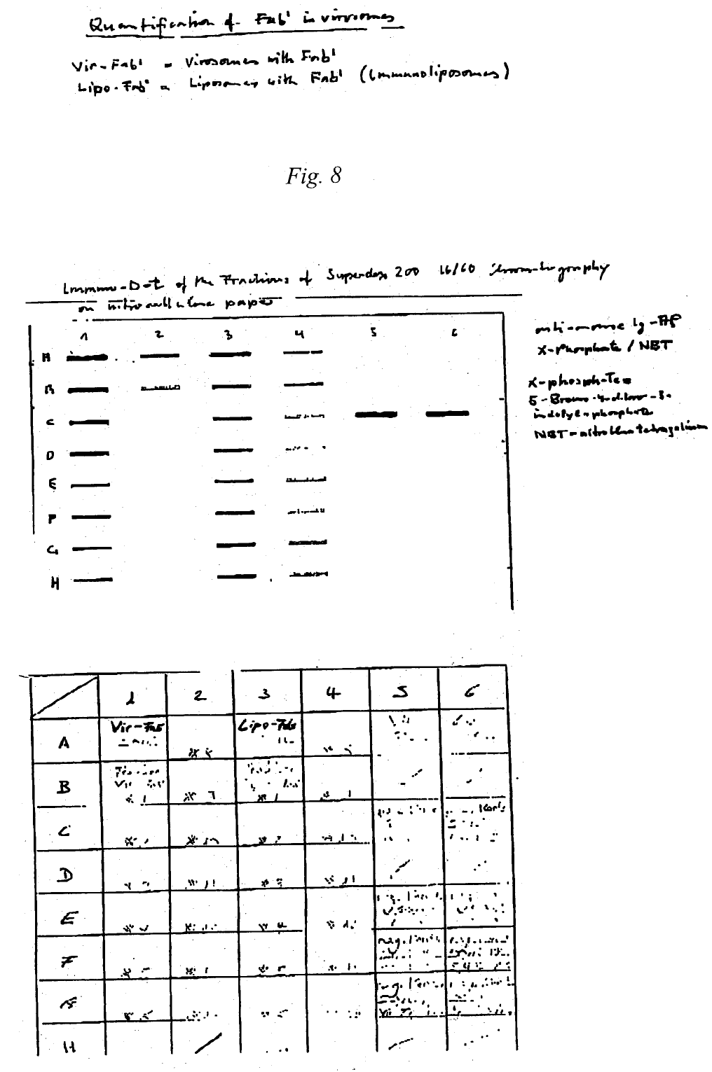

FIG. 9 shows the purification of conjugated virosomes and quantification of Fab′ fragments.

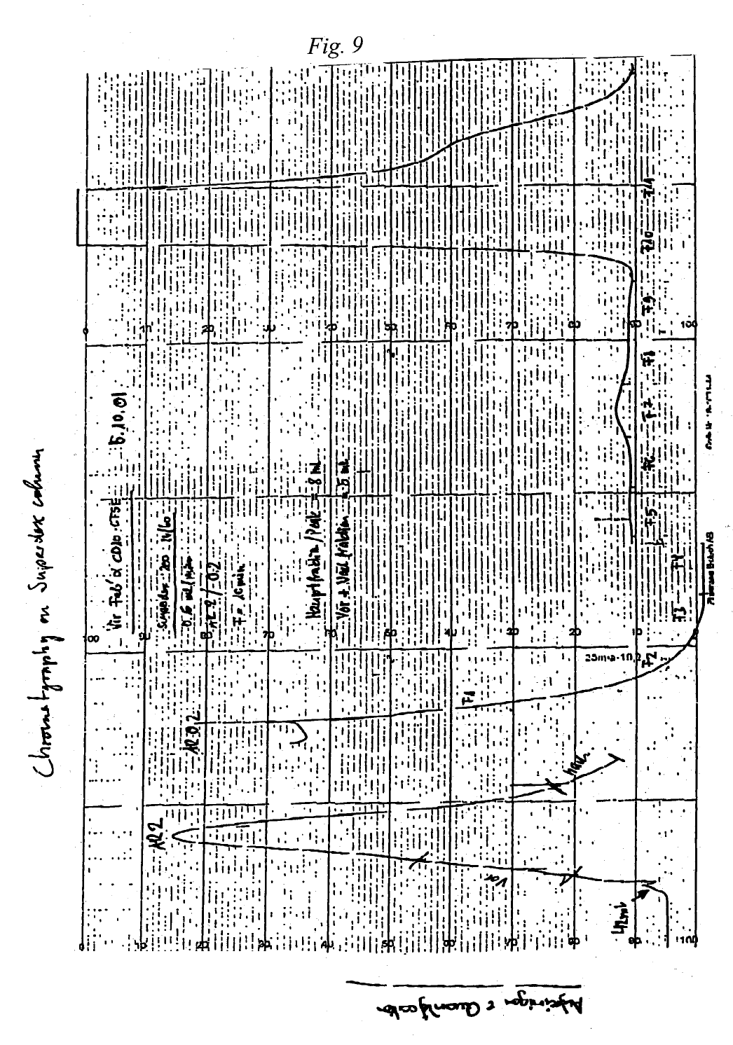

### DETAILED DESCRIPTION OF THE INVENTION

Accordingly, the present invention provides compositions for the effective delivery of therapeutic substances into the cytoplasm of targeted cells, as well as methods of producing the compositions, methods of delivery using the compositions, and methods of treating cancer.

In preferred embodiments of the invention, virosomes containing a chemotherapeutic drug are targeted to tumor cells by conjugating antibody fragments to the surface of the virosomse. Antibody targets that are overexpressed by tumors include, for example, CPSF, EphA3, G250/MN/CAIX, HER-2/neu, Intestinal carboxyl esterase, alpha-fetoprotein, M-CSF, MUC1, p53, PRAME, RAGE-1, RU2AS, Telomerase, WT1, among many others known in the art. In addition, antigens that are uniquely expressed by tumors are also suitable targets for antibodies. Such antigens include, for example, BAGE-1, GAGE-1 through 8, GnTV, HERV-K-MEL, LAGE-1, MAGE-1 through 12, NY-ESO-1/LAGE-2, SSX-2, TRP2/INT2 and others known in the art. The generation of monoclonal antibodies against any of these or other suitable targets is performed by methods, such as hybridoma technology, that are well known in the art. Isolation of antibody fragments, such as Fab′, or F(ab)₂, is a matter of routine for a person of skill in the art and can be performed by using published protocols such as those found in Harlow and Lane, Antibodies, A Laboratory Manual, Cold Spring Harbor Laboratory, (1988).

As an example of a preferred embodiment of the invention, Fab′ fragments of an anti-Neu (rNeu) monoclonal antibody are produced for conjugation to virosomes to selectively target rNeu (HER-2) overexpressing breast tumors. The human epidermal growth factor receptor 2 gene encodes a 185-kd protein (HER-2/neu, p1185^{HER2}) that is a cell surface receptor with tyrosine kinase activity. The HER-2/neu receptor is expressed on the cell membrane of a variety of epithelial cell types and regulates aspects of cell growth and division through binding of specific growth factors. HER-2/neu is expressed at low levels in many normal cells, but overexpressed in a variety of cancers, including breast, ovarian, endometrial, gastric, pancreatic, prostate and salivary gland cancers (Hynes and Stern, Biochim Biophys Acta 1198: 164-184, 1994). Approximately 30% of metastatic breast cancers have been shown to overexpress HER-2/neu (Slamon et al., Science 244: 707-712, 1989). This overexpression is associated with a poor prognosis for the breast cancer patient, as it corresponds to decreased relapse-free periods and shortened survival time (O'Reilly et al., Br J Cancer 63: 444-446, 1991).

For the production of the HER-2/neu antibody fragments, hybridoma cells producing monoclonal antibody against HER-2/neu are used. The monoclonal antibody is purified through a Protein G column. F(ab)₂ is produced by partial digestion with pepsin and further reduced to Fab′ fragments under nitrogen in 30 mM cysteine, 100 mM Tris, pH 7.6, for 20 min at 37° C. as previously described by Shahinian and Silvius, Biochim Biophys Acta 1239: 157-167, 1995. Fab′ contains a free thiol group for coupling to the maleimido group of the flexible spacer arm, NHS-PEG-MAL.

The clinical implications of HER-2/neu overexpression in tumors have made HER-2/neu an attractive target for antibody-mediated immunotherapy as an adjunct to conventional chemotherapy. However, there are several disadvantages to conventional systemic chemotherapy that are not alleviated by the concomitant use of immunotherapeutic approaches. One of the greatest limitations of cancer chemotherapy are the severe side effects accompanying the use of some of the most broadly active antitumor agents. For example, anthracycline anticancer compounds, such as doxorubicin, have a very wide spectrum of anticancer activity, but their side effects, when administered systemically, include significant myelosuppression, gastrointestinal toxicity with acute nausea and vomiting, local tissue necrosis that may require skin grafting in some cases, and dose-dependent cardiotoxicity often resulting in irreversible cardiomyopathy with serious congestive heart failure. A new drug delivery system for cytotoxic drugs that can target the drug specifically to tumor cells would not only eliminate these side effects but also increase the effectiveness of the drug against the tumor by preventing drug absorption by other tissues.

Because reconstituted fusion-active viral envelopes (virosomes) are capable of binding to and penetrating into tumor cells, they represent a promising system for antibody or antibody fragment mediated tumor targeting of chemotherapeutic substances. A virosome loaded with a chemotherapeutic drug can, for example, be targeted against HER-2/neu to deliver the drug to HER-2 overexpressing tumor cells. Chemotherapeutic drugs are well known in the art and may be selected from the folate antagonists, such as methotrexate, aminopterin, 110-EDAM, trimetrexate, piritrexim, D1694, ralitrexed, lometrexol etc., pyrimidine and purine antimetabolites such as fluorouracil, pyrazofurin, 6-azauridine, 5-ethynyluracil, allopurinol, acivicin, 6-mercaptopurine, thioguanine, deoxycoformicin, 5-fluoroadenine arabinoside-5′-phosphate, 2-chlorodeoxyadenosine, hydroxyurea, etc.; alkylating agents and platinum antitumor compounds such as nitrogen mustards, aziridines and epoxides, alkyl sulfonates, nitrosoureas, triazenes, hydrazenes and related compounds, hexamethylmelamine etc.; anthracyclines and DNA intercalators, epipodophyllotoxins, DNA topoisomerases, aspariginase, microtubule-targeting drugs and many more (Cancer Medicine, e. 5, American Cancer Society, B. C. Decker Inc. 2000, Hamilton, London).

In a preferred embodiment of the present invention, the antibody fragment is linked to a flexible spacer arm by site-directed conjugation. The flexible spacer arm is designed to keep the antigen binding site of the targeting moiety on the surface of the virosome available for binding to the target cell. By this method, the antibody molecules are placed in a position which allows their binding potential to remain available. For this purpose, 100 mg of NHS-PEG-MAL containing a long polyethylene glycol spacer arm (PEG) are dissolved in 3 ml of anhydrous methanol containing 10 μl of triethylamine. Then, 45 mg of dioleoyl phosphatidylethanolamine dissolved in 4 ml of chloroform and methanol (1:3;v/v) are added to the solution. The reaction is carried out under nitrogen for 3 h at room temperature (RT). Methanol/chloroform is removed under decreasing pressure and the products are redissolved in chloroform. The solution is extracted with 1% NaCl to remove unreacted material and water-soluble by-products. The PE-PEG-MAL is further purified by silic acid chromatography as described by Martin et al. (1982), with some modifications: the silica gel column has a diameter of 1.5 cm and is loaded with 14 sislical gel (Kieselgel 60, Fluka 60752). Elution is performed with the following gradient: Chloroform:methanol 29:1, 28:2, 27:3, 26:4 (ml) etc. 6 ml fractions are collected. PEG-PEG-MAL is obtained in fractions 13-31. Fractions and purity of PE-PEG-MAL are analyzed by TLC on silicon with chloroform-methanol-water 65:25:4. PE-PEG-MAL is dissolved in Tris-HCl buffer (100MM, pH 7.6) containing 10 mg/150 μl of octaethylenglycol-monododecylether (C₁₂E₈). To this solution the Fab′-fragments are added at a Fab′/PE-PEG-MAL ratio of 1:10. The solution is stirred at RT for 2 hr under nitrogen. Further C₁₂E₈ is added to obtain a 1%-C₁₂E8-solution and the reaction mixture is stirred overnight at 4° C. Unreacted PE-PEG-MAL is removed by the addition of 400 μl of washed, moist Thiopropyl Sepharose 6B. After a 3-hour incubation, the gel is removed by centrifugation. PE-PEG-Fab′-solution (3.6 ml) is sterilized by passage through a 0.2-μm filter and stored as a 0.01 M C₁₂E8 detergent solution.

In another preferred embodiment, Fab′ fragments of the targeting antibody, here an anti-rNeu monoclonal antibody (mAb), are conjugated to virosomes in a procedure that avoids precipitation of the conjugated virosomes. This method allows the reliable and consistent preparation of highly purified conjugated virosomes targeted to specific cells and is suitable for the large scale production of targeted virosomes. A further advantage of this method of preparation is the control over the quantity of targeting moieties per virosome. Hemagglutinin (HA) from the A/Singapore/6/86 strain of influenza virus is isolated as described in Waelti and Glueck, Int. J. Cancer 77: 728-733, 1998. Supernatant containing solubilized HA trimer (2.5 mg/ml) in 0.01M C₁₂E₈ detergent solution is used for the production of virosomes. Phospatidylcholine (38 mg) in chloroform is added to a round-bottom flask and the chloroform evaporated by a rotary evaporator to obtain a thin PC (phosphatidylcholine) film on the glass wall. The supernatant (4 ml containing 10 mg HA) and 3.6 ml of PE-PEG-Fab′ (containing 4 mg Fab′-fragments) from Example 3 are added to this flask. Under gentle shaking, the PC film covering the glass wall of the flask is solubilized by the C₁₂E₈ detergent containing mixture. The detergent of the resulting solution is removed by extraction with sterile Biobeads SM-2. The container is shaken for 1 hr by a REAX2 shaker (Heidolph, Kelheim, Germany). To remove the detergent completely, this procedure is repeated three times with 0.58 mg of Biobeads, after which a slightly transparent solution of Fab′-Virosomes is obtained. Quantitative analysis reveals that 1 ml of Fab′-Virosomes contain 1.3 mg of HA, 5 mg of PC and 0.53 mg of Fab′-fragments. Concentrations of Fab′ are determined by an immunoassay of the fractions collected from the gel filtration on the High Load Superdex 200 column as described in Antibodies, A Laboratory Manual. The procedure for the production of virosomes without Fab′ is the same except that no PE-PEG-Fab′ is added. The FITC-virosomes and Fab′-FITC virosomes are prepared by the same method, with the exception that carboxyfluorescein N-succinimidyl ester coupled to PE is built into the lipid bilayer.

In a preferred embodiment, conjugated virosomes are provided that allow maximal binding of the targeting moiety to cells without steric interference by virosomal membrane proteins. The advantage of using virosomes as a drug delivery system is the intrinsic capability of influenza virus to enter any mammalian cells triggered by its hemagglutinin. This effect is conserved if the native hemagglutinin is inserted into the virosomal lipid bilayer (Stegman et al., EMBO 6:2651-2659, 1987; Waelti and Glueck, Int. J. Cancer 77: 728-733, 1998). Therefore, in a preferred embodiment of the invention, the targeting antibody fragments, here anti-rNeu-Fab′ fragments, are conjugated to the virosomal surface (which contains many hemagglutinin proteins) by a linker with a long polyethylene glycol spacer arm, thus enabling virosomes to target the therapeutic drug, such as rNeu, to cancer cells expressing the targeted antigen, free of hindrance by other surface proteins.

In another preferred embodiment, the method of conjugation of antibody fragments to the virosome is performed by first producing the long flexible spacer arm precursor (PE-PEG-MAL), then reacting the Fab′ fragments with the long spacer to produce PE-PEG-Fab′. With this method, the flexible spacer arm is linked to the antibody fragment in such a way as to keep the antibody binding site freely available for binding, and finally inserting the PE-PEG-Fab′ into the pre-formed virosomes. By this new and efficient method of preparation the undesirable precipitation of the virosomes is avoided and functional conjugated virosomes with bioactive antibody fragments on their surface are invariably produced. A further advantage of this method is the ability to control the number of targeting moieties per virosome. This method allows for precise quantification of the Fab′ fragments or other ligands and for control over the rate of entry of virosomes into targeted cells.

In another preferred embodiment, the conjugated virosomes are loaded with a therapeutic composition of interest. For cancer therapeutic applications of the present invention, any chemotherapeutic drug would be suitable for encapsulation by the virosomes. The methods and compositions of the present invention are further adaptable to any therapeutically relevant application that benefits from the targeted delivery of substances to specific cells and tissues. Such applications may include the targeted delivery of anticancer drugs to cancer cells, antiviral drugs to infected cells and tissues, antimicrobial, and anti-inflammatory drugs to affected tissue, as well as the delivery of therapeutics to only those organs and tissues that are affected by the particular disease, thereby increasing the therapeutic index of the therapeutic drug and avoiding systemic toxicity. For example, in tumor therapy, doxorubicin, an antitumor antibiotic of the anthracycline class, may be delivered by the methods and compositions of the present invention. Anthracyclines have a wide spectrum of antitumor activity and exert pleiotropic effects on the cell. Although they are classic DNA intercalating agents, their mechanism of cytotoxicity is thought to be related to interaction with the enzyme topoisomerase II with production of double-stranded DNA breaks and possibly to the generation of intracellular free radicals that are highly cytotoxic. Thus, the conjugated virosomes are loaded with doxorubicin to selectively in order to efficiently inhibit tumor progression of established rNeu overexpressing breast tumors. Doxorubicin is loaded into virosomes through a proton gradient generated by virosome-entrapped ammonium sulfate as described by Gabizon et al., J. Natl. Cancer Inst. 81: 1484-1488, 1989. To load virosomes with ammonium sulfate, an ammonium sulfate solution (4.17 g/ml) is added to the virosome solution (7.5 ml), sonicated for 1 min and dialysed (Spectra/Por 2.1, Biotech DispoDialyzers, MWCO: 15'000, Spectrum Medical Industries, Houston, Tex., USA) against 1 liter of PBS containing 5% of glucose for 24 hours at 4° C. After 24 hours the dialysis buffer is changed and the virosome solution dialyzed for a futher 24 hours. To prepare the doxorubicin loading solution, 10 mg of doxorubicin is dissolved in 3 ml of water and sterilized through a 0.2-μm filter, then 750 μl of sterile 5× concentrated PBS and 5% glucose are added.

The virosome solution and doxorubicin loading solution are warmed to 33° C., then 2 volumes of virosome solution are mixed with 1 volume of doxorubicin loading solution. The mixture is incubated for 10 h at 33° C. and further incubated overnight at 28° C. Non-encapsulated doxorubicin is separated from the virosomes by gel filtration on a High Load Superdex 200 column (Pharmacia, Uppsala, Sweden), equilibrated with sterile PBS, 5% glucose. The void volume fractions containing Fab′-virosomes with encapsulated doxorubicin are eluted with 5% glucose in PBS and collected.

The amount of encapsulated drug, in this case, doxorubicin, is determined by absorbance at 480 nm. Virosome preparations contain on average 150 μg/ml doxorubicin. The mean diameter of the virosomes is determined by photon-correlation spectroscopy (PCS) with a Coulter N4Plus Sub-Micron-Particle Size Analyzer (Miami, Fla., USA). The proper expression of viral fusogenic activity of the virosomes is measured as previously described by Hoekstra et al., Biochemistry 23: 5675-5681, 1984, by an assay based on octadecylrhodamine (R18) fluorescence dequenching.

In another preferred embodiment, the conjugated loaded virosomes are tested for their ability to inhibit cell proliferation and to kill tumor cells. Cytotoxic activities of the conjugates are tested by a sodium 3′-(1-phenylaminocarconyl-3,4-terazolium)-bis(4-methoxy-6-nitro) benzene sulfonic acid hydrate (XTT) assay for measuring cell proliferation, as described by Jost et al., J. Immunol. Methods 147: 153-165, 1992. Briefly, cells (10,000) of both cell lines are seeded in 96 well-plate overnight in DMEM with 10% FCS. Cells are then cultured in fresh medium with various concentrations of anti-rNeu antibody (7.16.4), Fab′-Vir and empty Vir for 48 h. XTT solution is added according to the manufacturer's description (Roche Diagnostics, Rotkreuz, Switzerland). After 4 h of incubation at 37° C., the optical density is measured using an ELISA reader. Each value represents a mean±SEM of 3 samples. The Fab′-Doxo-Virosomes clearly combine the antiproliferative properties of the mAb and the cytotoxic effect of doxorubicin.

In another preferred embodiment, the conjugated virosomes loaded with a cytotoxic drug are used to inhibit tumor growth and effect tumor regression. Tumor implantation and therapeutic treatment are performed after anesthesia with i.p. injection of medetomidine hydrochloride (Domitor, Orion, Espoo, Finland, 500 μg/kg body weight), climazolamum (Climasol, Gräub, Bern, Switzerland, 5 mg/kg) and fentanyl citrate (Fentanyl-Janssen, Janssen-Cilag, Baar, Switzerland, 50 μg/kg). Mice are shaved at the injection sites and rNeu⁺ (NF 9006) and rNeu- (M/BB659) cells at a concentration of 2×10⁶ are injected s.c. Treatment is started when palpable tumors of at least 5 mm in diameter have formed. Injections into the tail vein of 200%1 of Doxorubicin-Virosomes (Doxo-Vir, doxorubicin 150 μg/ml), Fab′-Doxorubicin-Virosomes (Fab′-Doxo-Vir, same doxorubicin concentration, Fab′ at 182 μg/ml), Fab′-Vir (Fab′ at 182 μg/ml) and free Doxorubicin at a concentration of 150 μg/ml are performed 3 times a week for the whole observation period. Tumors are measured every 3-4 days measuring the length and width of each tumor with vernier calipers. Tumor volume is calculated using the Furthermore, Fab′-Doxo-Virosomes significantly inhibit tumor formation at a tumor load representing metastatic spread. These results indicate that virosomes conjugated with an antibody against a tumor antigen are a promising new selective drug delivery system for the treatment of tumors expressing a specific tumor antigen.

In another preferred embodiment of the invention, conjugated virosomes are used to arrest the metastatic spread of tumors. HER-2/neu overexpression occurs in the primary tumor as well as in metastatic sites (Niehans et al., J. Natl. Cancer Inst. 85: 1230-1235, 1993). 4D5, a murine mAb directed against the extracellular domain of human HER-2/neu protein, has been shown to elicit receptor internalization and ultimately to inhibit proliferation of HER-2/neu overexpressing breast cancer cells in vitro and in breast cancer xenografts (Baselga et al., Cancer Res. 58: 2825-2831, 1998). Another anti-HER-2/neu mAb (clone 7.16.4), initially raised against the ectodomain of rat Neu (rNeu), was shown to share an epitope with 4D5 and to inhibit tumor formation of HER-2/neu overexpressing tumor cells (Zhang et al., Exp. Mol. Pathol. 67: 15-25, 1999). The effect of Fab′ conjugated virosomes on the growth of tumor metastases is assessed as follows. For the micrometastatic stage of tumor formation, 0.2×10⁶ tumor cells are injected s.c. and treatment is started 3-5 days later. Again, different virosome combinations are compared in mice injected with free Doxorubicine and controls. After 9 injections (over 3 weeks) the treatment is stopped in all groups. Tumor formation is assessed by palpation followed by measurement of tumor size as described in the previous example. Tumor formation is defined as tumor size beyond possible regression (90 mm³).

In another preferred embodiment, a method of selectively destroying tumor cells is provided. Tumor-targeted virosomes loaded with cytotoxic compounds can be administered to a subject with a tumor burden. The virosomes selectively target the cells expressing a surface marker to which the targeting moiety conjugate on the virosomal surface binds. Upon binding, the virosome enters the cell and rapidly releases its cytotoxic contents, thereby destroying the targeted cells.

In another preferred embodiment, a method of producing tumor regression is provided. Tumors that express unique surface proteins or overexpress certain cell surface markers can be effectively targeted with conjugated virosomes containing cytotoxins. Upon binding to the tumor cells, the virosomes release their drug contents into the tumor cell cytoplasm, thereby destroying the tumor cells and inducing the regression of the tumor.

### Results

Binding and internalization of Fab′-virosomes (Fab′-Vir) to rNeu⁺ tumor cells was analyzed and compared it to unconjugated virosomes. The present invention demonstrates the cytotoxic effect of Fab′-Virosomes in vitro. Furthermore, the methods and compositions of the present invention show a significant inhibition of tumor progression in mice with established tumors treated with doxorubicin encapsulated in Fab′-Virosomes (Fab′-Doxo-Vir). The same virosomal construct is capable of successfully inhibiting tumor formation in mice through early treatment after tumor inoculation. These results demonstrate that virosomes conjugated with a tumor-specific antibody are a new and efficient drug delivery system with tumor specific targeting. Anti-rNeu Fab′ virosomes bind to murine breast cancer cells and are efficiently internalized.

The produced virosomes (reconstituted fusion-active viral envelopes), composed of a single phospholipid bilayer and densely covered with hemagglutinin (HA) spikes, are relatively homogenous in size, ranging from 80-200 nm. The PE-PEG-MAL spacer is used to conjugate anti-rNeu Fab′ fragments by their free thiol group to the maleimido group. The long polyethylene glycol spacer arm allows an extended and site-directed binding of Fab′-molecules, and thus prevents any potential blockage of the antigen binding sites by the neighboring HA trimers. The chosen ratio of total phospholipid/PE-PEG-Fab′ results in 100-150 Fab′ fragments per virosome.

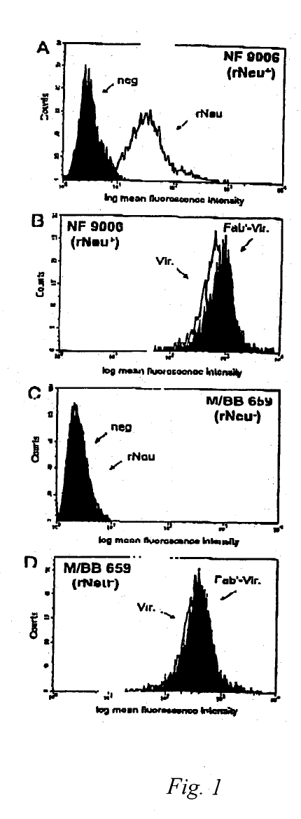

To be most effective as a drug-delivery system, virosomes should bind to tumor targets. FITC-labeled, unconjugated virosomes (Vir) and FITC-labeled virosomes conjugated with Fab′-fragments of an anti-rNeu mAb (Fab′-Vir) were analyzed for their binding capacity to breast cancer cell lines. As depicted in the FACS histograms in FIG. 1A, the NF9006 (rNeu⁺) breast cancer cell line expressed significant levels of rNeu on the cell surface in comparison to the negative rNeu expression of M/BB 659 (FIG. 1C) breast cancer cells when the specific anti-rNeu mAb (clone 7.16.4) was used. In contrast to the specific binding with anti-rNeu mAb, virosomes showed an increased binding to breast tumor cells independent of their expression of rNeu on the cell surface. Cells overexpressing rNeu had an augmented binding of Fab-Vir compared to the unconjugated FITC-labeled Vir (FIG. 1B). Although Vir and Fab′-Vir showed a strong binding to rNeu⁻ breast cancer cells, there was no difference between Fab′-Vir and Vir (FIG. 1D). These results indicated that virosomes were capable of binding to the tumor cell membranes and that Fab′-Vir showed an increased binding to rNeu⁺ breast cancer cell lines through the specific binding of anti-rNeu Fab′-fragments.

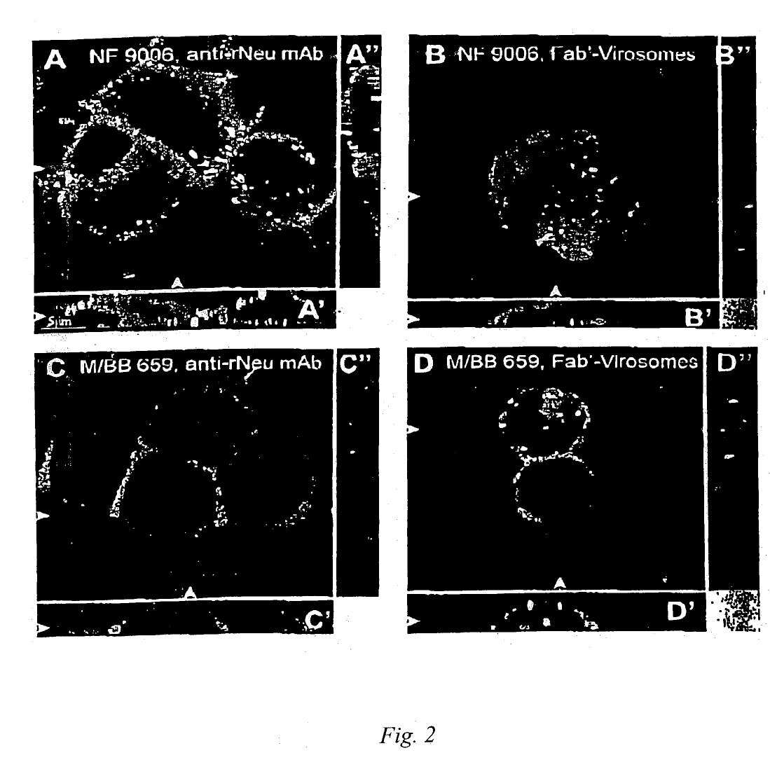

Confocal laser scanning microscopy (CLSM) was used to study the internalization and distribution of FITC-conjugated virosomes in tumor cells after binding. NF9006 and M/BB 659 breast cancer cells were incubated at 37° C. for 1 h with FITC-conjugated Fab′-Vir and Vir. CLSM with a 3D reconstruction showed that in both cell lines, bound virosomes were efficiently internalized as evidenced by the large aggregates of FITC-fluorescence observed within the breast cancer cell line (FIGS. 2B+2D). There was no visible difference in internalization between Fab′-Vir and Vir (data not shown). By contrast, in NF9006 cells, FITC-conjugated anti-rNeu mAb (clone 7.16.4) was predominantly localized in the membrane or in its close proximity (FIG. 2A), whereas no FITC-fluorescence was observed in M/BB 659 tumor cells (FIG. 2C). These results suggest that virosomes may represent a novel carrier system to deliver encapsulated drugs into the cytosol of solid tumors.

In Vitro Cytotoxicity Studies of Fab′-Vir.

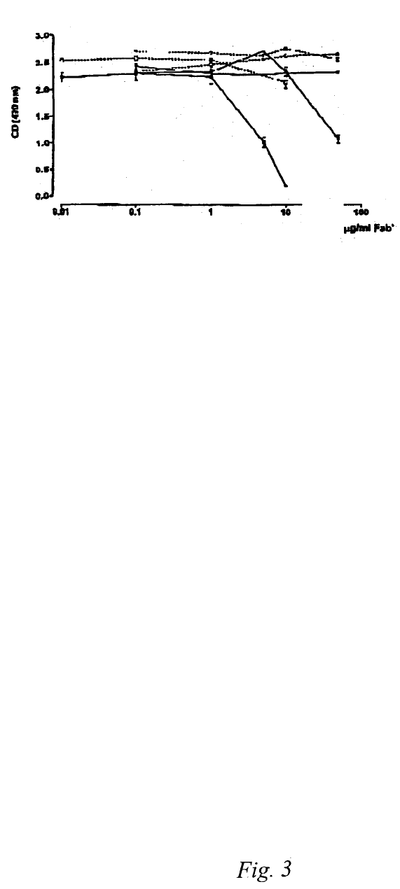

To clarify whether virosomes containing anchored Fab′-fragments of an anti-rNeu mAb might have an antiproliferative activity, we investigated the effect of empty virosomes (Vir), Fab′-Vir and anti-rNeu mAb on breast cancer cells in vitro. Cells were cultured in the presence of increasing concentrations of anti-rNeu mAb, Fab′-Vir and Vir and proliferation of the cells was assessed by the colorimetric XTT assay (10). As shown in FIG. 3, neither rNeu⁺ nor rNeu⁻ breast cancer cells were affected in their proliferation where different concentrations of empty Vir were added to the cultures. In contrast, mAb 7.16.4 was capable of specifically inhibiting proliferation of rNeu⁺ breast cancer cells in a dose dependent manner, whereas the rNeu⁻ breast cancer cells were only marginally affected in their proliferation. Monovalent Fab′-fragments are known to be much less effective in the inhibition of proliferation (Park et al., PNAS 92: 1327-1331, 1995), however the conjugation of monovalent anti-rNeu Fab′-fragments to the surface of virosomes showed an important antiproliferative effect on rNeu⁺ breast cancer cells. Whereas the addition of 10 μg/ml of intact anti-rNeu mAb induced >90% growth inhibition, the addition of 50 μg/ml of Fab′-Vir was necessary to induce a 50% proliferation inhibition. The anti-proliferative effect of Fab′-Vir was specific for rNeu⁺ cells as no inhibitory effect was seen in cultures with rNeu⁻ breast cancer cells. Since Vir showed no inhibitory or toxic effect on breast cancer cells in vitro and Fab′-Vir exhibited a dose dependent inhibition of proliferation of rNeu⁺ cells, we conclude that the virosomal lipid envelope with inserted HA was not cytotoxic to breast cancer cells. Treatment of established tumors.

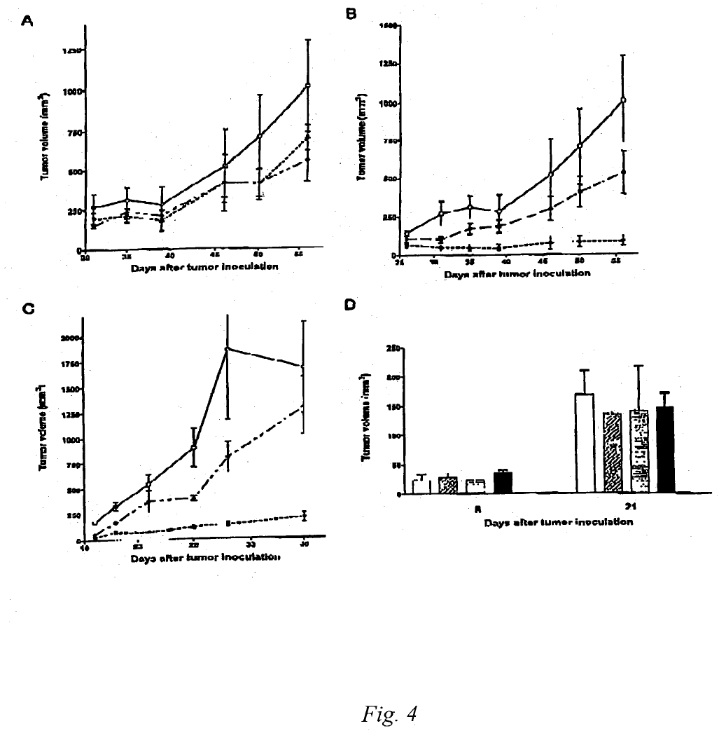

A first set of experiments was designed to clarify whether the observed in vitro binding and internalization of virosomes into tumor cells also corresponded to an enhanced delivery of encapsulated cytotoxic drugs in vivo. The therapeutic effect on breast tumor implants of i.v. injected free Doxorubicin (free Doxo) was compared to doxorubicin encapsulated in virosomes (Doxo-Vir). In a second set of experiments the specific drug-targeting with immunovirosomes (Fab′-Doxo-Vir) was tested for an increased therapeutic effect. Mice were inoculated s.c. with 2×10⁶ rNeu⁺ and rNeu⁻ tumor cells. After tumor formation (5 mm diameter) treatment was started with i.v. injections of either free Doxo, Doxo-Vir, Fab′-Doxo-Vir and Fab′-Vir every 3-4 days. Control groups received no treatment. As shown in FIG. 4A there was a marginally significant decrease in tumor progression over time in mice treated with Doxo-Vir compared to the control groups. However, there was no significant difference in tumor progression in mice treated with free Doxo or Doxo-Vir. In contrast, as shown in FIGS. 4B and 4C, tumor progression was almost completely inhibited in the group of mice treated with the Fab′-Doxo-Vir, that was significantly more effective than Doxo-Vir. To determine whether the effect of Fab′-Doxo-Vir was dependent on targeting doxorubicin to the tumor cells or on the antibody blocking effect of the Fab′-fragments, mice with established tumors were also treated with Fab′-Vir. There was a significant inhibition on tumor progression when Fab′-Doxo-Vir were used for treatment compared to the group of mice where only Fab′-Vir were injected, demonstrating an additive effect of targeting doxorubicin to the tumor cells by Fab′ fragments on virosomes (FIG. 4C).

The specificity of rNeu targeting was further assessed by treating mice with established rNeu⁺ (M/BB 659) tumors with the different virosome compounds. As shown in FIG. 4D the treatment of mice with Doxo-Vir, Fab′-Vir and Fab′-Doxo-Vir had no significant effect on rNeu³¹ tumor progression over time as compared to untreated control groups. These results demonstrated that in all experiments rNeu⁺ tumor growth was specifically and efficiently suppressed in mice treated with Fab′-Doxo-Vir as compared to mice treated with free Doxo, Doxo-Vir and Fab′-Vir.

Cytotoxicity of Anti-rNeu Immunovirosomes Containing Doxorubicin.

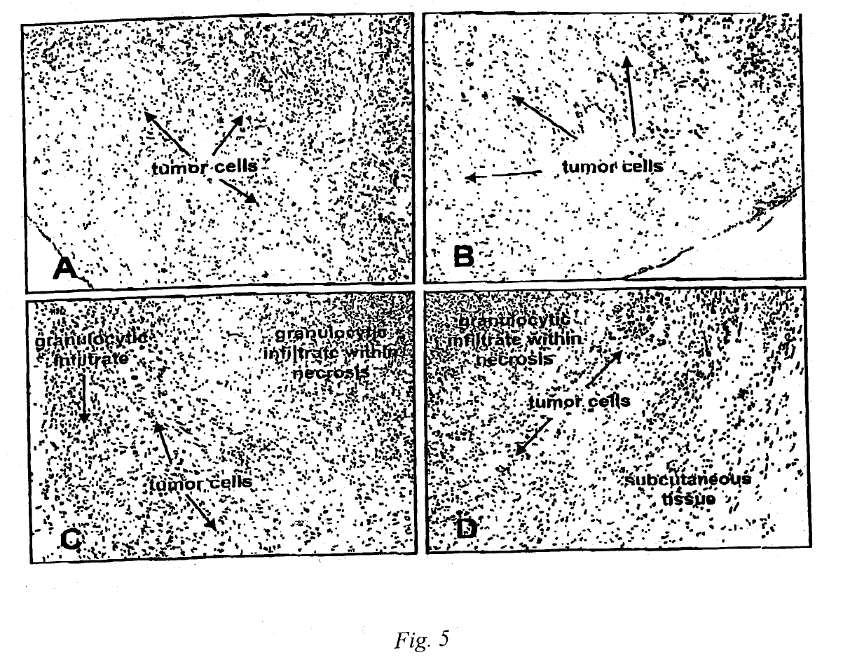

To get further insight into the histological pattern induced by the different virosome treatments on inocculated tumors, histopathological sections were prepared at different time points after tumor injection. At day 5-7 after rNeu⁺ breast cancer cell implantation, tumors were excised and analyzed. In untreated mice, the border of the tumor was well demarcated from the neighboring subcutaneous normal tissue (FIG. 5A). Tumor cells appeared uniform with a large, slightly granular cytoplasm and some nuclei showed mitosis, but no inflammatory infiltrates were detected. In mice treated with Doxo-Vir, again the tumor border was well delineated from the underlying normal tissue and there was hardly any inflammatory infiltrate in the surrounding subcutaneous tissue nor any necrotic tumor cells visible (FIG. 5B). In contrast, in mice treated with Fab′-Vir a large amount of necrotic cells was found predominantly in the center of the tumors (FIG. 5C). The surviving tumor cells appeared in contiguous groups surrounded by a significant infiltrate of granulocytic cells also infiltrating the underlying subcutanous tissue. In mice treated with Fab′-Doxo-Vir, moreover, most of the tumor cells were necrotic and replaced by an inflammatory infiltrate composed mainly of granulocytes and eosinophils. There was also an impressive granulocytic infiltrate in the vicinity of surviving tumor conglomerates (FIG. 5D). In contrast, mice injected with the rNeu⁻ breast cancer cell line (M/BB 659) showed no necrosis in any of the virosome treated animals and some inflammatory infiltration surrounding the tumors were mainly seen in Fab′-Vir and Fab′-Doxo-Vir treated mice (data not shown). These results confirmed the effect seen in vivo by Fab′-Doxo-Vir treated mice on established rNeu⁺ tumors.

Treatment of Recently Implanted Tumors.

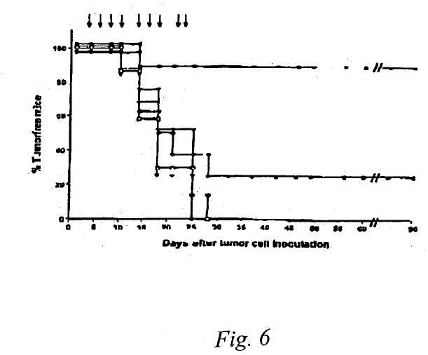

As an alternative evaluation of the efficacy of the virosomal carrier system provided by the present invention, the longtime protection from tumor formation in animals with recently implanted tumors was investigated. Therefore, treatment was started 3-5 days after s.c. injection of 2×10⁵ Neu⁺ breast cancer cells into mice. All mice were treated with 9 injections of virosomal compounds for 3 weeks and the tumor formation was monitored in the following weeks. Within 4 weeks after tumor cell inoculation all mice of the control group, groups treated with free Doxo and Doxo-Vir developed tumors and had to be sacrificed due to excessive tumor load (FIG. 6). The median time to tumor formation in mice treated with Doxo-Vir was 20 days and did not significantly differ from mice treated with free Doxo (20 days) or the control group (17 days)(p>0.4-0.8). Whereas mice treated with Fab′-Vir had no significant (p>0.3) difference in the median time to tumor formation compared to the control group, we noticed that 20% of mice did not develop tumors during the observation period of >90 days. Mice treated with Fab′-Doxo-Vir showed a significant increase (p<0.005) in time to tumor formation with 90% of mice having no tumor at >90 days after tumor cell inoculation. These data suggested that Fab′-Doxo-Vir are highly efficient in delivering cytotoxic drugs to tumor cells and preventing tumor formation in recently implanted tumors.

The data presented here demonstrates that virosomes can be used as a new drug carrier system and be selectively targeted to breast tumors to deliver cytotoxic drugs. Antibody fragment conjugated virosomes, such as anti-rNeu virosomes (Fab′-Vir), have the advantage to bind to the ubiquitous sialic acid residues on the cell surface by HA and retained the binding capacity to rNeu receptors by Fab′ fragment of the mAb (clone 7.16.4). The data of FACS analysis demonstrated that site-directed conjugation of Fab-fragments to a crosslinker with a long polyethylene glycol spacer arm, resulted in specific binding of Fab′-Vir to rNeu expressing cancer cells. By confocal microscopy it was demonstrated that virosomes bound to tumor cells became rapidly internalized. In contrast, internalization of targeted liposomes (immunoliposomes) were previously shown to occur slowly dependent upon the cell surface density of HER-2/neu and the internalization rate of receptor after crosslinking with anti-HER-2/neu mAb (Kirpotin et al., Biochemistry 36: 66-75, 1997). The Fab′-fragments of the anti-rNeu mAb, retained the antiproliferative effect when coupled to virosomes and were linked to the ability to cause receptor internalization (Zhang et al., Exp. Mol. Pathol. 67: 15-25, 1999). So far, evidence for an in vivo cytotoxic effect of doxorubicin-containing liposomes was mostly based on the release of drugs from liposomes into the extracellular space (Horowitz et al., Biochim Biophys Acta 1109: 203-209, 1992; Naessander et al. Biochim Biophys Acta 1235: 126-129, 1995). Internalizing epitopes, such as HER-2/neu, are thought to be more efficient at increasing the intracellular drug concentration as entry of the drug into the cells is not only dependent on passive diffusion. However, the appropriate selection of mAb and the targeted antigen are crucial for the success of intracellular drug delivery by liposomes. Virosomes, in contrast, are rapidly and efficiently internalized through receptor-mediated endocytosis and are trapped in the endosomes where a pH change from 5-6 triggers the fusion of the virosomal membrane with the endosomal membrane. As a consequence, the virosome encapsulated doxorubicin is delivered with high efficiency into the cytoplasm of the targeted cells.

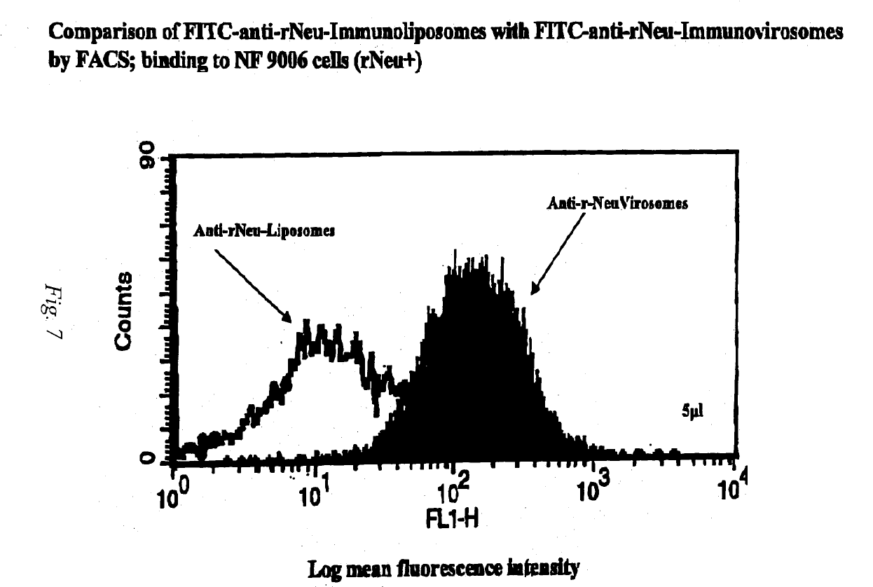

The in vivo efficacy of virosomes loaded with doxorubicin (Fab′-Doxo-Vir) demonstrated a significant decrease of tumor progression in large established tumors compared to tumor-bearing mice receiving free Doxo or Doxo-Vir (drug-loaded, non-targeted virosomes). The specificity and the increased therapeutic index of Fab′-Doxo-Vir in vivo on rNeu- established tumors thus exemplifies the efficacy of the drug delivery system provided by the present invention. The inability of immunoliposomes to obliterate established tumors was attributed in part to the specific binding to cell surface receptors at the periphery of the tumor and the ineffectiveness of liposomes to penetrate tumor cells (Sugano et al., Canc. Res. 60: 6942-6949, 2000; Moase et al., Biochim Biophys Acta 1510: 43-55, 2001). The present invention thus demonstrates that virosomes have a higher capacity to enter tumor cells (FIG. 7) and can induce tumor regression. Furthermore, the treatment with targeted virosomes of small poorly vascularized tumors prevented the formation of established tumors. Thus, treatment with virosomes is effective in halting or reversing early metastatic spread and can be combined with other tumor reducing modalities, such as surgery, radiotherapy and chemotherapy.

The present invention also provides some impact with respect to animal models for studying anticancer therapeutics. So far, experiments mainly in SCID mice were used for testing anti-HER-2/neu coated liposomal compounds (Park et al, PNAS 92: 1327-1331, 1995). Considerable differences exist between the tolerated dose of doxorubicin between SCID mice (2-3 mg/kg) and normal mice (6 mg/kg) (Williams et al., Cancer Res. 53: 3964-3967, 1993). As SCID mice have a defect in DNA repair mechanisms, these models may not be the appropriate mouse models to test immunotherapeutic strategies. The present invention uses an immune competent mouse strain and syngeneic breast tumor cell lines derived from the same transgenic mouse to demonstrate the effectiveness of targeted virosomes containing therapeutic drugs (Morrison and Leder, Oncogene 9: 3417-3426, 1994). In these experiments, mice were injected with 7.5 mg/kg virosomal doxorubicin, a dose required for successful therapeutic outcome.

Virosomes have therapeutic potential and this invention provides specific targeting of therapeutic virosomes by coupling Fab′ fragments to virosomal membranes. The virosomes of the invention are efficiently internalized by target cells and the encapsulated drug delivered to the tumor cells. Thus, the targeted virosomes will find widespread applicability in drug therapies in which cell or tissue-specific drug delivery is advantageous.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. Unless otherwise specified, general cloning procedures, such as those set forth in Sambrook et al., Molecular Cloning, Cold Spring Harbor Laboratoy (2001), Ausubel et al. (eds.) Current Protocols in Molecular Biology, John Wiley & Sons (2000), and Harlow and Lane, Antibodies, A Laboratory Manual, Cold Spring Harbor Laboratory, (1988) are used. One skilled in the art may develop equivalent means or reactants without the exercise of inventive capacity and without departing from the scope of the invention.

It will be understood that many variations can be made in the procedures herein described while still remaining within the bounds of the present invention.

### Example 1

Chemicals, Cell Lines and Animals

Octaethylene lycol mono (n-dodecyl) ether (C₁₂E₈), 3-sn-phosphatidylcholine solution (PC), 5(6)-carboxyfluorescein N-succinimidyl ester and doxorubicin.HCl are available from Fluka (Buchs, Switzerland). 1,2-Dioleoyl-sn-glycero-3-phosphoethanolamine (PE) is sold by Avanti Polar-Lipids (Alabaster, Ala., USA). Bio-Beads SM-2 are available from BioRad (Richmond, Calif., USA). NHS-PEG-MAL, MW 2,000 (N-hydroxysuccinimidyl (maleimido) polyethylene glycol) are available from Shearwater Polymers (Huntsville, Ala., USA); PEG chains having a mean molecular weight (MW) of 2000 contain 45 monomers.

MMTV/r-Neu: FVB mice transgenic for the rat neu protein (rNeu-TG) are available from Charles River, Germany. The rNeu⁺ breast-cancer cell line NF 9006, derived from a rNeu-TG mouse and the rNeu⁻ breast cancer cell line M/BB 659, derived from a c-myc-TG mouse have been described previously.

### Example 2

Production of Antibody Fragments

Hybridoma cells producing monoclonal antibody are obtained from M. Greene. The monoclonal antibody is purified through a Protein G column. F(ab)₂ is produced by partial digestion with pepsin and further reduced to Fab′ fragments under nitrogen in 30 mM cysteine, 100 mM Tris, pH 7.6, for 20 min at 37° C. as previously described by Shahinian and Silvius, Biochim Biophys Acta 1239: 157-167, 1995. Fab′ contains a free thiol group for coupling to the maleimido group of the flexible spacer arm, NHS-PEG-MAL.

### Example 3

Method of Constructing the Virosome Targeting Ligand and Spacer

This example demonstrates the site-directed conjugation of the Fab′ fragment to the flexible spacer arm designed to keep the antigen binding site available for binding to the target cell. In order to place the Fab′ molecules in a position which allows their bivalent binding potential to remain available, Fab′-fragments are conjugated to the flexible spacer arm by site-directed conjugation. 100 mg of NHS-PEG-MAL containing a long polyethylene glycol spacer arm (PEG) are dissolved in 3 ml of anhydrous methanol containing 10 μl of triethylamine. Then, 45 mg of dioleoyl phosphatidylethanolamine dissolved in 4 ml of chloroform and methanol (1:3;v/v) are added to the solution. The reaction is carried out under nitrogen for 3 h at room temperature (RT). Methanol/chloroform is removed under decreasing pressure and the products are redissolved in chloroform. The solution is extracted with 1% NaCl to remove unreacted material and water-soluble by-products. The PE-PEG-MAL is further purified by silic acid chromatography as described by Martin et al. (1982), with some modifications: the silica gel column has a diameter of 1.5 cm and is loaded with 14 sislical gel (Kieselgel 60, Fluka 60752). Elution is performed with the following gradient: Chloroform:methanol 29:1, 28:2, 27:3, 26:4 (ml) etc. 6 ml fractions are collected. PEG-PEG-MAL is obtained in fractions 13-31. Fractions and purity of PE-PEG-MAL are analyzed by TLC on silicon with chloroform-methanol-water 65:25:4. PE-PEG-MAL is dissolved in Tris-HCl buffer (100 mM, pH 7.6) containing 10 mg/150 μl of octaethylenglycol-monododecylether (C₁₂E₈). To this solution the Fab′-fragments are added at a Fab′/PE-PEG-MAL ratio of 1: 10. The solution is stirred at RT for 2 hr under nitrogen. Further C₁₂E₈ is added to obtain a 1%-C₁₂E₈-solution and the reaction mixture is stirred overnight at 4° C. Unreacted PE-PEG-MAL is removed by the addition of 400 μl of washed, moist Thiopropyl Sepharose 6B. After a 3-hour incubation, the gel is removed by centrifugation. PE-PEG-Fab′-solution (3.6 ml) is sterilized by passage through a 0.2-μm filter and stored as a 0.01 M C₁₂E₈ detergent solution.

### Example 4

Method of Producing of FAB′ Virosomes

This example demonstrates the preparation of conjugated virosomes targeted to specific cells. Hemagglutinin (HA) from the A/Singapore/6/86 strain of influenza virus is isolated as described in Waelti and Glueck, Int. J. Cancer 77: 728-733, 1998. Supernatant containing solubilized HA trimer (2.5 mg/ml) in 0.01M C₁₂E₈ detergent solution is used for the production of virosomes. Phospatidylcholine (38 mg) in chloroform is added to a round-bottom flask and the chloroform evaporated by a rotary evaporator to obtain a thin PC (phosphatidylcholine) film on the glass wall. The supernatant (4 ml containing 10 mg HA) and 3.6 ml of PE-PEG-Fab′ (containing 4 mg Fab′-fragments) from Example 3 are added to this flask. Under gentle shaking, the PC film covering the glass wall of the flask is solubilized by the C₁₂E₈ detergent containing mixture. The detergent of the resulting solution is removed by extraction with sterile Biobeads SM-2. The container is shaken for 1 hr by a REAX2 shaker (Heidolph, Kelheim, Germany). To remove the detergent completely, this procedure is repeated three times with 0.58 mg of Biobeads, after which a slightly transparent solution of Fab′-Virosomes is obtained. Quantitative analysis reveals that 1 ml of Fab′-Virosomes contain 1.3 mg of HA, 5 mg of PC and 0.53 mg of Fab′-fragments. Concentrations of Fab′ are determined by an immunoassay of the fractions collected from the gel filtration on the High Load Superdex 200 column as described in Antibodies, A Laboratory Manual. The procedure for the production of virosomes without Fab′ is the same except that no PE-PEG-Fab′ is added.

The FITC-virosomes and Fab′-FITC virosomes are prepared by the same method, with the exception that carboxyfluorescein N-succinimidyl ester coupled to PE is built into the lipid bilayer.

### Example 5

Encapsulation of Doxorubicin into Virosomes

This example demonstrates the loading of a therapeutic composition into the Fab′ conjugated virosomes. Doxorubicin is loaded into virosomes through a proton gradient generated by virosome-entrapped ammonium sulfate as described by Gabizon et al., J. Natl. Cancer Inst. 81: 1484-1488, 1989. To load virosomes with ammonium sulfate, an ammonium sulfate solution (4.17 g/ml) is added to the virosome solution (7.5 ml), sonicated for 1 min and dialysed (Spectra/Por 2.1, Biotech DispoDialyzers, MWCO: 15'000, Spectrum Medical Industries, Houston, Tex., USA) against 1 liter of PBS containing 5% of glucose for 24 hours at 4° C. After 24 hours the dialysis buffer is changed and the virosome solution dialyzed for a futher 24 hours. To prepare the doxorubicin loading solution, 10 mg of doxorubicin is dissolved in 3 ml of water and sterilized through a 0.2-μm filter, then 750 μl of sterile 5× concentrated PBS and 5% glucose are added.

The virosome solution and doxorubicin loading solution are warmed to 33° C., then 2 volumes of virosome solution are mixed with 1 volume of doxorubicin loading solution. The mixture is incubated for 10 h at 33° C. and further incubated overnight at 28° C. Non-encapsulated doxorubicin is separated from the virosomes by gel filtration on a High Load Superdex 200 column (Pharmacia, Uppsala, Sweden), equilibrated with sterile PBS, 5% glucose. The void volume fractions containing Fab′-virosomes with encapsulated doxorubicin are eluted with 5% glucose in PBS and collected.

The amount of encapsulated doxorubicin is determined by absorbance at 480 nm. Virosome preparations contain on average 150 μg/ml doxorubicin. The mean diameter of the virosomes is determined by photon-correlation spectroscopy (PCS) with a Coulter N4Plus Sub-Micron-Particle Size Analyzer (Miami, Fla., USA). The proper expression of viral fusogenic activity of the virosomes is measured as previously described by Hoekstra et al., Biochemistry 23: 5675-5681, 1984, by an assay based on octadecylrhodamine (R18) fluorescence dequenching.

### Example 6

### Assessment of Binding and Internalization of FAB′ Conjugated Virosomes

This example demonstrates how the ability of the conjugated virosomes to bind to and enter the target cells is assessed by immunofluorescent labeling. To assess binding of the targeted virosomes, rNeu⁺ (positive) and rNeu⁻ (negative, not expressing rNeu) breast cancer cell lines NF 9006 and M/BB 659, respectively, are incubated at 4° C. for 30 min with FITC-conjugated anti-rNeu-virosomes (Fab′-Vir) and FITC-virosomes (Vir). The fluorescence is analyzed by flow cytometry on a FACScan (Becton Dickinson, Heidelberg, Germany).

To assess internalization of the virosomes, cells in suspension are incubated with 1 μg/ml FITC conjugated Fab′-Vir or Vir for 30 min at 4° C. and then washed in PBS. Internalization is performed in 1 ml complete medium for 1 h at 37° C. Control samples are incubated at 4° C. for 30 min with anti-neu mAb Ab-4 (Oncogene Science, Tarzana, Calif., USA), then washed with PBS, followed by incubation with FITC-conjugated goat anti-mouse IgG (Southern Biotechnology, Birmingham, Ala., USA). Internalization is performed for 1 hour at 37° C. in medium. Samples are washed and fixed-in 3% paraformaldehyde. Fixed cells are permeabilized in 0.2% Triton X-100 for 15 min and stained for F-Actin with rhodamine-phalloidin (1:100, Molecular Probes, Leiden, Netherlands). After staining the cells are centrifuged on slides and preparation mounted in PBS:glycerol (2:1, Calbiochem, Lucerne, Switzerland).

For visualization of virosome binding and internalization by confocal microscopy, Microradiance system from BioRad combined with an inverted Nikon microscope (Eclipse TE3000) is used (Lasers: Ghe/Ne 543 nm and Ar 488 nm). Optical sections at intervals of 0.3 μm are taken with a 100×/1.4 Plan-Apochromat objective. Image processing is done on a Silicon Graphics workstation using IMARIS, a 3D multi-channel image processing software (Bitplane AG, Zurich, Switzerland).

### Example 7

Assessment of the Cytotoxicity of Targeted Virosomes

This example demonstrates the effect of the delivery of Fab′ conjugated virosomes containing doxorubicin on target cell proliferation. Cytotoxic activities of the conjugates are tested by a sodium 3′-(1-phenylaminocarconyl-3,4-terazolium)-bis (4-methoxy-6-nitro) benzene sulfonic acid hydrate (XTT) assay for measuring cell proliferation, as described by Jost et al., J. Immunol. Methods 147: 153-165, 1992. Briefly, cells (10,000) of both cell lines are seeded in 96 well-plate overnight in DMEM with 10% FCS. Cells are then cultured in fresh medium with various concentrations of anti-rNeu antibody (7.16.4), Fab′-Vir and empty Vir for 48 h. XTT solution is added according to the manufacturer's description (Roche Diagnostics, Rotkreuz, Switzerland). After 4 h of incubation at 37° C., the optical density is measured using an ELISA reader. Each value represents a mean±SEM of 3 samples.

### Example 8

Treatment of Established Tumors

This example demonstrates the effect of the conjugated virosomes on tumor growth in vivo. Tumor implantation and therapeutic treatment are performed after anesthesia with i.p. injection of medetomidine hydrochloride (Domitor, Orion, Espoo, Finland, 500 μg/kg body weight), climazolamum (Climasol, Gräub, Bern, Switzerland, 5 mg/kg) and fentanyl citrate (Fentanyl-Janssen, Janssen-Cilag, Baar, Switzerland, 50 μg/kg). Mice are shaved at the injection sites and rNeu⁺ (NF 9006) and rNeu⁻ (M/BB659) cells at a concentration of 2×10⁶ are injected s.c. Treatment is started when palpable tumors of at least 5 mm in diameter have formed. Injections into the tail vein of 200 μl of Doxorubicin-Virosomes (Doxo-Vir, doxorubicin 150 μg/ml), Fab′-Doxorubicin-Virosomes (Fab′-Doxo-Vir, same doxorubicin concentration, Fab′ at 182 μg/ml), Fab′-Vir (Fab′ at 182 μg/ml) and free Doxorubicin at a concentration of 150 μg/ml are performed 3 times a week for the whole observation period. Tumors are measured every 3-4 days measuring the length and width of each tumor with vernier calipers. Tumor volume is calculated using the formula: π/6×largest diameter×(smallest diameter)².

### Example 9

Effect of Conjugated Virosomes on Recently Implanted Tumors

This example demonstrates the effect of Fab′ conjugated virosomes on the growth of tumor metastases. For the micrometastatic stage of tumor formation, 0.2×10⁶ tumor cells are injected s.c. and treatment is started 3-5 days later. Again, different virosome combinations are compared in mice injected with free Doxorubicine and controls. After 9 injections (over 3 weeks) the treatment is stopped in all groups. Tumor formation is assessed by palpation followed by measurement of tumor size as described in the previous example. Tumor formation is defined as tumor size beyond possible regression (90 mm³).

We claim:

1. A method of making a targeted synthetic membrane vesicle for the delivery of therapeutic substances to selected cells and tissues comprising the steps of:
(a) linking a spacer molecule with an antibody fragment;
(b) conjugating the spacer/antibody fragment of step (a) to a virosome.

2. The method of claim 1, wherein said spacer molecule is a flexible spacer arm.

3. The method of claim 1, wherein said spacer molecule is a polyethylene glycol spacer arm.

4. The method of claim 1, wherein said antibody fragment is a Fab′ fragment.

5. The method of claim 1, wherein said antibody fragment derives from an antibody against a tissue-specific antigen.

6. The method of claim 5, wherein said tissue-specific antigen is an antigen overexpressed or specifically expressed by tumors.

7. The method of claim 6, wherein said tissue-specific antigen is selected from the group consisting of CPSF, EphA3, G250/MN/CAIX, HER-2/neu, Intestinal carboxyl esterase, alpha-fetoprotein, M-CSF, MUC1, p53, PRAME, RAGE-1, RU2AS, Telomerase, WT1, BAGE-1, GAGE-1 through 8, GnTV, HERV-K-MEL, LAGE-1, MAGE-1 through 12, NY-ESO-1/LAGE-2, SSX-2, TRP2/INT2

8. The method of claim 7, wherein said tissue-specific antigen is HER-2/neu.

9. The method of claim 1, wherein said linking is performed by site-directed conjugation.

10. The method of claim 9, wherein said site-directed conjugation positions the antibody fragment so as to make the antigen binding site available for binding to the target cell.

11. The method of claim 1, wherein said conjugation of said spacer/antibody fragment to said virosome is accomplished without precipitation of the conjugated virosomes.

12. The method of claim 1, further comprising the step of loading the virosome with a therapeutic composition of interest.

13. The method of claim 12, wherein said therapeutic composition of interest is selected from the group consisting of anticancer, antiviral, antimicrobial, and anti-inflammatory drugs.

14. A composition comprising a virosome conjugated to an antibody fragment.

15. The composition of claim 14, wherein the antibody fragment is conjugated to said virosome by a flexible spacer molecule.

16. The composition of claim 15, wherein said spacer molecule is a polyethylene glycol spacer arm.

17. The composition of claim 14, wherein said antibody fragment is a Fab′ fragment.

18. The composition of claim 14, wherein said antibody fragment derives from an antibody against a tissue-specific antigen.

19. The composition of claim 18, wherein said tissue-specific antigen is an antigen overexpressed or specifically expressed by tumors.

20. The composition of claim 19, wherein said tissue-specific antigen is selected from the group consisting of CPSF, EphA3, G250/MN/CAIX, HER-2/neu, Intestinal carboxyl esterase, alpha-fetoprotein, M-CSF, MUC1, p53, PRAME, RAGE-1, RU2AS, Telomerase, WT1, BAGE-1, GAGE-1 through 8, GnTV, HERV-K-MEL, LAGE-1, MAGE-1 through 12, NY-ESO-1/LAGE-2, SSX-2, TRP2/INT2

21. The composition of claim 20, wherein said tissue-specific antigen is HER-2/neu.

22. The composition of claim 14, wherein the virosome encapsulates a therapeutic composition of interest.

23. The composition of claim 22, wherein said therapeutic composition of interest is selected from the group consisting of anticancer, antiviral, antimicrobial, and anti-inflammatory drugs.

24. A method of killing a tumor cell, comprising administering to a subject the composition of claim 22.
